(19) United States
(12) Patent Application Publication
(10) Pub. No.: US 2011/0230645 A1
(43) Pub. Date: Sep. 22, 2011

(54) PURIFICATION OF FACTOR V

(75) Inventors: Maartje Verschuur, Leiden (NL); Evert Heemskerk, Leiden (NL); Paul Henri Markus, Loosdrecht (NL); Gerardus Jacobus Van Mierlo, Almere (NL)

(73) Assignee: CRUCELL HOLLAND B.V.

(21) Appl. No.: 13/131,001

(22) PCT Filed: Dec. 15, 2009
(86) PCT No.: PCT/EP09/67159

§ 371 Date: May 24, 2011

Related U.S. Application Data
(60) Provisional application No. 61/201,942, filed on Dec. 16, 2008.

(30) Foreign Application Priority Data
Dec. 16, 2008 (EP) 08171792.8

(51) Int. Cl. C07K 1/36; C07K 1/18

(52) U.S. Cl. 530/381

(57) ABSTRACT

The invention provides methods for purifying blood coagulation Factor V from biological fluids.

### CROSS-REFERENCE TO RELATED APPLICATIONS

This is a national phase entry under 35 U.S.C. §371 of International Patent Application PCT/EP2009/067159, filed Dec. 15, 2009, published in English as International Patent Publication WO 2010/069946 A1 on Jun. 24, 2010, which claims the benefit under Article 8 of the Patent Cooperation Treaty to European Patent Application Serial No. 08171792.8, filed Dec. 16, 2008, and under 35 U.S.C. §119(e) to U.S. Provisional Patent Application Ser. No. 61/201,942, filed Dec. 16, 2008.

### TECHNICAL FIELD

The invention relates to the field of proteins, in particular to the purification of proteins. More in particular, the invention relates to the purification of blood coagulation Factor V (FV).

### BACKGROUND

Factor V is a major player in the coagulation cascade leading to the formation of the fibrin clot through the action of thrombin. Activated Factor V (FVa) acts as a cofactor for the serine protease Factor Xa (FXa), enhancing conversion of prothrombin to thrombin by five orders of magnitude. The half-life of FVa is down-regulated by activated protein C (APC) that cleaves FVa at several postions in the heavy chain of the protein. Cleavage at two postions, R306 and R506, was shown to be the major contributor to the inactivation of FVa by APC. The APC-resistant double mutant of FV, FV-Leiden/Cambridge that harbors the R306T and R506Q mutations, increases the lifetime of this protein and, therefore, is a potential therapeutic candidate in treatment of blood disorders involving low thrombin generation (WO2008/059009).

Full-length FV is a 330 KDa polypeptide with a domain structure similar to Factor VIII (FVIII). After proteolytic activation by thrombin to form FVa, the protein is composed of a heavy chain and a light chain non-covalently associated with a calcium binding site at the interface between the two chains. FV contains multiple post-translational modifications such as glycosylations, sulfations and phosphorylations (qualitatively represented by black dots in FIG. 1) that are important for the cofactor function. The glycosyl groups give about 13% of the total molecular weight of FV with a high degree of sialylation.

Research grade protocols for the purification of Factor V have been described by several authors, both for the purification of Factor V from human or bovine plasma (Neshheim et al.) and for the purification of recombinant Factor V produced on cell cultures (V. D. Neut et al., Bos et al.). In general, these protocols have not been designed with the purpose of pharmaceutical production, and cannot readily be used for this goal. For instance, the protocol described by Bos et al. contains the following steps:
1. Concentration of cell culture harvest using an “artificial kidney” (Hemoflow F5 hollow fiber from Fresenius, Bad Homburg, Germany).
2. Purification of Factor V by immuno-affinity using a monoclonal antibody specific for Factor V directed against the B-domain (a-FV); performed in batch mode.
3. Buffer exchange by dialysis.
4. Further concentration by anion exchange chromatography.
5. Buffer exchange into the 50% glycerol-based storage buffer by dialysis.

The main drawbacks of this protocol are the long process time and the fact that the protocol includes process steps that are not suitable for scale-up (i.e., initial concentration step, buffer exchange step by dialysis). Other protocols disclosed hitherto have similar drawbacks.

There remains a need in the art for further possibilities to purify Factor V. More specifically, there remains a need for industrial processes for Factor V purification. It is the object of the present invention to provide alternative methods for purification of Factor V.

### DISCLOSURE

The invention provides a method for purifying coagulation Factor V from a biological fluid comprising, in the given order, the steps of: a) binding factor V to an anion exchanger; b) washing the anion exchanger with a first solution to remove contaminants; c) eluting Factor V; d) specifically binding factor V to a matrix containing anti-Factor V antibodies; e) washing the matrix containing anti-Factor V antibodies with a second solution to remove contaminants; and f) eluting Factor V.

In one embodiment, the anion exchanger used in step a) is a filter. In another embodiment, the anion exchanger used in step a) is a chromatographic monolith containing groups with high anion exchanger functionality, such as quaternary amine groups.

In further embodiments, the elution in step c) is performed by treating the anion exchanger with a buffer containing between 0.3 and 1 M NaCl.

In further embodiments, the matrix used in step d) is an immuno-affinity capture filter membrane. In preferred embodiments, the matrix used in step d) is a cross-linked polystyrene-divinylbenzene matrix to which epoxide functional groups are bound.

The invention further provides the use of a chromatographic monolith containing quaternary amine groups for the purification of coagulation Factor V from a biological fluid.

The invention further provides the use of a chromatographic monolith containing quaternary amine groups for the separation of active Factor V from inactive Factor V, wherein the active form of Factor V is at least two times as active as the inactive form of Factor V in a clot activity assay.

In certain embodiments, the coagulation Factor V has been recombinantly expressed. In further embodiments, the coagulation factor V is an APC-resistant Factor V mutant.

### BRIEF DESCRIPTION OF THE DRAWINGS

FIG. 1: Domain structure of Factor V molecule.

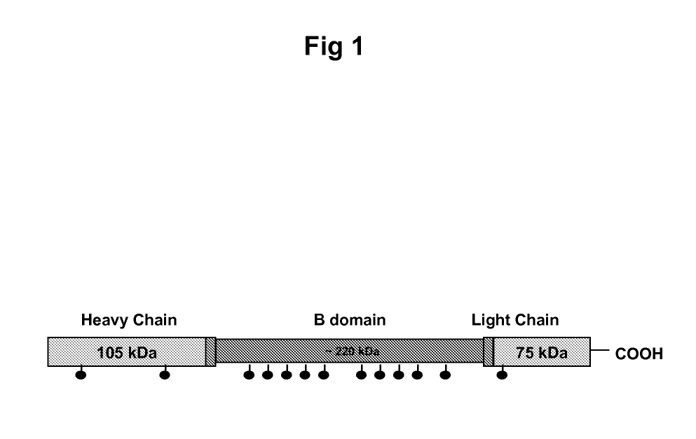

FIG. 2: Schematic overview of the clot activity assay.

FIG. 3: Process flow diagram of Factor V purification process based on research-grade protocol described by Bos et al.

FIG. 4: Process flow diagram of Factor V purification process according to the invention (anion exchange followed by immuno-affinity).

FIG. 5: Process flow diagram of Factor V purification process according to the invention with the use of a chromatographic monolith.

FIG. 6: SDS-Page of eluate fractions from the anion exchanger (monolith) and the immuno-affinity step.

### DETAILED DESCRIPTION OF THE INVENTION

The present invention provides a scalable process for Factor V purification with a reduced process time compared to processes developed hitherto. The invention resides in the use of an anion exchange step followed by an immuno-affinity step. This particular order allows for high flow capacity and a reduced process time compared to previously designed processes such as, for instance, the process described by Bos et al. Moreover, the current process enables the separation of active Factor V from inactive Factor V.

Compared to Bos et al., the novel process of the present invention is initiated with an anion exchange (AEX) step, wherein factor V is concentrated. Also, during this step, DNA, as well as contaminants, are removed from the biological fluid. Subsequently, either directly or following a dilution, the biological fluid is further processed on an immuno-affinity matrix containing anti-Factor V antibodies.

Surprisingly, this resulted in the possibility to omit the initial concentration step, as well as the dialysis step, from the process described in Bos et al. and therewith, a significant time reduction could be achieved. The omission of the concentration and dialysis steps, which are steps that are not scalable, allow for the current process of this invention to be scaled-up and, therefore, to be applicable in an industrial process, in contrast to the process in Bos et al.

In one embodiment of the present invention, the anion exchange step is performed with a specific chromatographic monolith, which has a high resolution. A two-step gradient elution allows for the separation of active Factor V from inactive Factor V.

Several properties of Factor V, as well as the possibility to use it for hemostatic treatment, are, for instance, described in WO2008/059009, incorporated herein in its entirety by reference. The term “Factor V,” as used herein, not only encompasses within its meaning human Factor V, such as wild-type human plasma Factor V (SEQ ID NO:1 in WO2008/059009), as well as natural allelic variations thereof, but also Factor V variants containing one or more amino acid alterations by deletion, substitution or addition, and chemically modified Factor V. Several examples of such molecules are given in WO2008/059009. For instance, one or more amino acids of Factor V, preferably not more than 30, more preferably not more than 20, still more preferably not more than 10, most preferably zero, one, two or three amino acids, may be replaced with other amino acids, or eliminated, or added. Typically, molecules that are at least 70%, preferably at least 80%, more preferably at least 90%, still more preferably at least 95% identical in amino acid sequence to SEQ ID NO:1 of WO2008/059009 is encompassed within the term Factor V as used herein. In certain embodiments, Factor V has the amino acid sequence of wild-type human plasma Factor V. The term “Factor V” also includes APC-resistant FV mutants (Bertina et al. 1994, Svensson et al. 1994, Williamson et al. 1998 and Chan 1998). These mutants are inactivated more slowly by APC and, hence, prolong the activity of Factor Xa. Thus, via their effect on FXa, these FV mutants enhance thrombin formation. APC-resistant FV cannot act as a cofactor for APC and thus lacks the anti-coagulant effect of its wild-type counterpart. In preferred embodiments, Factor V according to the present invention, therefore, is APC-resistant Factor V (WO2008/059009, WO2008/059043).

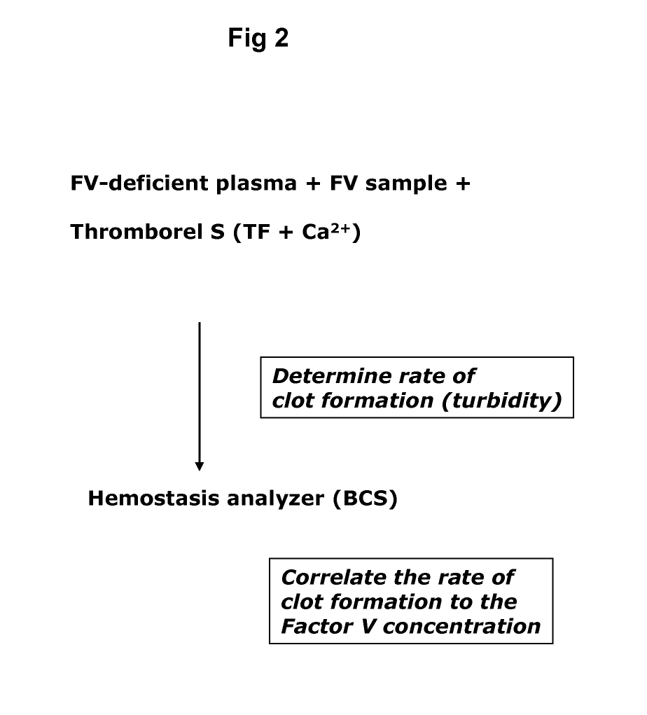

Factor V, according to the present invention, exists either in an active or inactive form. The active form induces clot formation while the inactive form is not capable of inducing clot formation. The ability to induce clot formation is tested in a clot activity assay, which is based on a prothrombin time (PT) assay performed using FV-deficient human plasma. The clot activity assay determines the concentration of active Factor V contained in a preparation by correlating the clotting time (time for clot formation induced by factor V) to the Factor V concentration. FIG. 2 shows a schematic view of the clot activity assay and Example 3 provides a suitable clot activity assay. The assay was performed according to methods known to the person skilled in the art.

Factor V is purified, according to the invention, from a biological fluid. A biological fluid may be any fluid derived from or containing cells, cell components or cell products. Biological fluids include, but are not limited to, cell cultures, cell culture supernatants, cell lysates, cleared cell lysates, cell extracts, tissue extracts, blood, plasma, serum, milk, urine, plant extracts and fractions thereof, all of which may also be homogenizates and filtrates, and fractions thereof, for instance, collected by chromatography of unfractionated biological fluids.

Factor V may be purified from a wide variety of biological fluids, including cell culture supernatants, which naturally produce Factor V, but preferably of cells that have been genetically modified to produce recombinant Factor V, such as mammalian cells, e.g., Chinese hamster ovary (CHO) cells, HEK293 cells, BHK cells, PER.C6® cells (as deposited at the ECACC under no. 96022940; for recombinant expression of proteins in PER.C6® cells; see, e.g., U.S. Pat. No. 6,855,544), yeast, fungi, insect cells, and the like, or prokaryotic cells, or transgenic animals or plants. In certain embodiments, recombinant expression is achieved in PER.C6® cells that further over-express a sialyltransferase, e.g., human α-2,3-sialyltransferase (see, e.g., WO2008/059043, WO2008/059009). Methods for recombinant expression of desired proteins are known in the art, and recombinant production of, for instance, APC-resistant FV has been described in, e.g., EP 0756638 and WO2008/059009.

In one embodiment, the biological material is derived from human blood plasma. In a preferred embodiment, the biological material is derived from a culture of cells in which Factor V is recombinantly expressed, for instance, a cell culture supernatant or cell-conditioned culture medium thereof.

Cell culture supernatants or cell-conditioned culture medium in the capture step of Factor V according to the invention may be derived from cells grown in the presence of serum, such as fetal calf serum, or grown in serum-free medium. Cell-conditioned culture medium denotes a nutrient medium in which cells have been cultured and that contains cell products. When working with biological fluids containing cells, cell debris and the like, it is preferred to first filter and/or (ultra)centrifuge the fluid to remove particulate contaminants. Recombinant Factor V is secreted by the cells into the cell culture medium (cell-conditioned culture medium), or present in cell lysates, and can be separated according to the invention from other cell components, such as cell waste products, cell debris and proteins or other collected material.

Purification of Factor V is the process of increasing the concentration of Factor V (enriching) in a sample in relation to other components of the sample, resulting in an increase of the purity of Factor V. The increase in purity of Factor V may be followed by use of methods known in the art, such as, for instance, by use of SDS-PAGE, HPLC or ELISA. Purification is done to remove undesired contaminants, and therewith increase the purity of Factor V. The term “purified” as used herein in relation to a protein does not refer only to absolute purity (such as a homogeneous preparation); instead, it refers to a protein that is relatively purer than in the natural environment. A step of purifying a protein thus relates to obtaining a protein preparation in which the protein is purer than in the biological material prior to the purification step, i.e., the preparation contains less contaminants, “contaminants” in this context including proteins other than Factor V.

The present invention provides a process for purification of Factor V, which comprises an anion exchange chromatography step followed by an immuno-affinity step.

Ion exchange chromatography (see, e.g., GE Healthcare, “Ion Exchange Chromatography & Chromatofocusing,” Principles & Methods, Cat. no. 11-0004-21) relies on charge interactions between the protein of interest and the ion exchange matrix, which is generally composed of a solid support, such as agarose, dextran, cross-linked cellulose, and the like, covalently bound to a charged group. Charged groups are classified according to type (cationic and anionic) and strength (strong or weak); the charge characteristics of strong ion exchange media do not change with pH, whereas with weak ion exchange media, sample loading and capacity can change owing to loss of charge at varying pH, preventing protein binding. Examples of commonly used charged groups include diethylaminoethyl (DEAE; weak anionic exchanger), carboxymethyl (weak cationic exchanger), quaternary ammonium (strong anionic exchanger), and methyl sulfonate (strong cationic exchanger). Other charged groups are available as well. Ion exchange resins selectively bind proteins of opposite charge; that is, a negatively charged resin will bind proteins with a positive charge and vice versa.

The technique in general takes place in five steps: equilibration of the column to pH and ionic conditions ideal for target protein binding; reversible adsorption of the sample to the column through counterion displacement; introduction of elution conditions that change the buffer's pH or ionic strength in order to displace bound proteins; elution of substances from the column in order of binding strength (weakly bound proteins are eluted first); and re-equilibration of the column for subsequent purifications. The skilled person can design ion exchange chromatography protocols such that the target protein is selectively bound to the column (allowing contaminants to pass through) or so that contaminants adsorb and the target protein is excluded. In addition to resins that can be used in batch or to prepare columns, ion exchange can also be performed using high throughput ion exchange membranes/filters (i.e., a charged membrane or filter that contains ion exchange groups). Such ion exchange filters are known in the art and are commercially available, e.g., from Pall (e.g., Mustang™ series) and from Sartorius (e.g., Sartobind series). Such filters can have advantages compared to ion exchange columns, for instance, in Example 2, it is shown that filters allow for higher flow rates, resulting in a reduced process time.

Therefore, in one preferred embodiment of the present invention, the anion exchanger is a filter containing a cellulose matrix with quaternary ammonium functional groups bound to it.

Currently, a new generation of columns for ion exchange chromatography has been developed based on the CIM® (Convective interaction media) technology. The technology relies on large inner channel diameter and convective mass transfer. The CIM® monolithic supports are based on a highly cross-linked porous monolithic polymer, such as polyglycidyl methacrylate-co-ethylene dimethacrylate or polystyrene-divinylbenzene polymers with well-defined, bimodal channel-size distribution, which allow for high flow rates.

In one preferred embodiment of the present invention, the anion exchanger is a chromatographic monolith, which in certain embodiments is based on a polyglycidyl methacrylate-co-ethylene dimethacrylate matrix with quaternary amine (QA) functional groups. Herewith, the process time was reduced similarly as with the use of filters. Additionally, the chromatographic monolith could unexpectedly separate active Factor V from inactive Factor V, as shown in Example 3. The anion exchanger used in the present method is, therefore, preferably a chromatographic monolith containing quaternary amine groups. Such chromatographic monoliths are known in the art and commercially available, e.g., from BIA separations (e.g., CIM® QA monolithic column).

During the anion exchange chromatography step, the negatively charged Factor V proteins are bound to positively charged functional groups on the surface of the anion exchanger. This anion exchange chromatography step may be used for DNA removal as well. Since host cell DNA is negatively charged, it will be bound to functional groups on the surface of the chromatographic support.

Subsequent to the anion exchange chromatography step, the anion exchanger is washed with a buffer with high buffer capacity at pH value between about 5 and 9, preferably at pH value between about 6 and 8 (e.g., Tris, HEPES). In certain embodiments of the present invention, the buffer contains between 100 mM and 200 mM NaCl; between 0.5 mM and 5 mM CaCl₂ and between 1% and 20% glycerol. In a preferred embodiment, the buffer contains 150 mM NaCl, 1 mM CaCl₂ and 10% glycerol. Additionally, the buffer contains between 5 and 50 mM benzamidine, which is a protease inhibitor. Preferably, the buffer contains 10 mM benzamidine. The anion exchanger is washed in order to remove impurities.

Elution of factor V from the anion exchanger is achieved, e.g., by increasing the ionic strength of the buffer, in particular, by increasing the sodium chloride concentration. The optimal sodium chloride concentration is dependent on the type of anion exchanger. The person skilled in the art is able to optimize the sodium chloride concentration in order to obtain maximal Factor V elution. In certain embodiments of the present invention, the NaCl concentration in the elution buffer lies between about 0.5 and 1 M NaCl, for instance, when the anion exchanger is a filter. Preferably, the NaCl concentration in the elution buffer is about 0.6 M when the anion exchanger is a filter. In other embodiments of the present invention, the NaCl concentration in the elution buffer lies between 0.30 and 1 M NaCl, for instance, when the anion exchanger is a chromatographic monolith. Unexpectedly, it appeared that when applying a two-step buffer gradient on the chromatographic monolith, active Factor V could be separated from inactive Factor V. In a certain embodiment of the present invention, the NaCl concentration in the buffer used to elute active Factor V is between about 0.35 and 0.5 M. Preferably, the concentration is between about 0.35 and 0.4 M. Even more preferably, the concentration is about 0.36 M. In yet another embodiment of the present invention, the NaCl concentration in the buffer used to elute inactive Factor V is between about 0.5 and 1 M. Preferably, the concentration is between about 0.5 and 0.6 M. Even more preferably, the concentration is about 0.56 M.

According to the present invention, a chromatographic monolith can be used for the separation of active from inactive Factor V. Preferably, a chromatographic monolith containing quaternary amine groups is used for the separation of active from inactive Factor V.

In certain embodiments, the active form of Factor V as purified using a monolith is at least two times as active as the inactive form of Factor V in a clot activity assay.

In a later stage, DNA is eluted from the anion exchanger. This process requires a higher ionic strength compared to Factor V proteins. Therefore, material eluted from an anion exchanger at ionic strength suitable for Factor V elution will not contain host cell DNA.

According to the present invention, the anion exchange step is followed by an immuno-affinity step. The previously eluted fraction of Factor V is brought into contact with an immuno-affinity chromatography matrix.

Immuno-affinity chromatography is a specialized form of affinity chromatography (see, e.g., Affinity Chromatography, Principles & Methods, GE Healthcare, Cat. no. 18-1022-29) and, as such, utilizes an antibody or antibody fragment as a ligand immobilized onto a solid support matrix in a manner that retains its binding capacity. Immuno-affinity chromatography relies on the highly specific interaction of an antigen with its antibody. The highly selective loops on the antibody surface capture the antigen with high affinity, while having little interaction with impurities and other components that may also be present in the biological fluid.

In the present invention, the immuno-affinity chromatography matrix contains anti-Factor V antibodies, which bind specifically to Factor V. In certain embodiments, the anti-Factor V antibodies are covalently bound to an epoxide functional group, which is coupled to the support matrix.

In certain embodiments of the present invention, the support matrix is an immuno-affinity capture filter membrane. These membranes can consist of a cellulose matrix with epoxide functional groups as, for instance, the Sartobind Epoxy 75 from Sartorius.

In one embodiment of the present invention, the support matrix is a cross-linked polystyrene-divinylbenzene matrix. In a preferred embodiment, the support matrix consists of cross-linked polystyrene-divinylbenzene flow-through particles. These particles are coated with a cross-linked polyhydroxylated polymer, which are then activated with epoxide functional groups. Cross-linked polystyrene-divinylbenzene matrix with epoxide groups are commercially available, e.g., from Applied Biosystems (e.g., POROS® series).

During the immuno-affinity chromatography step, the Factor V proteins are specifically bound to anti-Factor V antibodies. Subsequent to the immuno-affinity chromatography step, the immuno-affinity matrix is washed with a buffer, preferably at pH value between 6 and 8 (e.g., Tris, HEPES). In certain embodiments of the present invention, the buffer is a Tris buffer, which contains between 100 mM and 300 mM NaCl, between 0.5 mM and 5 mM CaCl₂ and between 5% and 20% ethylene glycol. In a preferred embodiment, the buffer consists of 200 mM NaCl, 1 mM CaCl₂ and 10% ethylene glycol. The immuno-affinity matrix is washed in order to remove impurities.

Elution of factor V from the immuno-affinity matrix was achieved, e.g., by increasing the ionic strength of the buffer, in particular, by increasing the sodium chloride concentration. The optimal sodium chloride concentration is dependent on the type of the immuno-affinity matrix. The person skilled in the art knows how to optimize the sodium chloride and ethylene glycol concentration in order to obtain maximal Factor V elution. In certain embodiments of the present invention, the NaCl concentration in the elution buffer is between about 1.5 M and 3 M. The ethylene glycol concentration lies between 40% and 60%. Preferably, the NaCl concentration in the elution buffer is 2 M and the ethylene glycol concentration is 50%.

In preferred embodiments, the Factor V purification process of the invention gives a yield of at least 15%, more preferred at least 20%, still more preferred at least 25%, still more preferred at least 30% of the amount of Factor V in the starting material. The eluted Factor V in preferred embodiments has a purity that is higher than 50%, preferably higher than 60%, more preferably higher than 70%, still more preferably higher than 80%, for instance, about 90% or higher.

Thus, in certain exemplary embodiments, the invention provides a method for purifying Factor V from a biological fluid, the method comprising a step of bringing a biological fluid comprising Factor V into contact with an anion exchanger, washing the anion exchanger to remove contaminants, and eluting Factor V by treating the anion exchanger with a buffer containing between 0.35 M and 1 M NaCl (depending on the type of anion exchanger) to obtain a preparation further enriched in Factor V. The method further comprises a step of bringing the preparation comprising Factor V into contact with an immuno-affinity matrix containing anti-Factor V antibodies to bind Factor V to the matrix, washing the matrix to remove contaminants and eluting Factor V to obtain a preparation further enriched in Factor V. The purity of Factor V in the purified Factor V preparation is preferably at least 90%, more preferably at least 95%.

Alternatively, the anion exchange step of the invention could be followed by a dilution step performed prior to the innuno-affinity step, for instance, to lower the salt concentration of the Factor V preparation in order to maximize Factor V binding to the immuno-affinity matrix.

According to the present invention, further purification steps may be performed after elution, following the immuno-affinity step. For example, the preparation can be, without limitations, further concentrated using conventional methods prior to buffer exchange to a final 50%-glycerol-based storage buffer or buffer exchange to a 5%-glycerol-based buffer and then freeze dried to obtain a final concentrated sample with a concentration of 50% glycerol.

It will be clear that different embodiments of the invention can be combined, for instance, a method is provided wherein the anion exchange step is performed with a filter and wherein the following immuno-affinity step is performed with a column containing a matrix to which Epoxide functional groups are bound. Also, a method is provided wherein the anion exchange step is performed with a chromatographic monolith and wherein the following immuno-affinity step is performed with a filter.

The practice of this invention will employ, unless otherwise indicated, conventional techniques of immunology, molecular biology, microbiology, cell biology, recombinant DNA, and protein purification, which are within the skill of the art. See, e.g., Sambrook, Fritsch and Maniatis, Molecular Cloning: A Laboratory Manual, 2^{nd} edition, 1989; Current Protocols in Molecular Biology, F. M. Ausubel et al., eds, 1987; the series Methods in Enzymology (Academic Press, Inc.); PCR2: A Practical Approach, M. J. MacPherson, B. D. Hams, G. R. Taylor, eds, 1995; Antibodies: A Laboratory Manual, Harlow and Lane, eds, 1988; Bioprocess Technology vol 9, Separation Processes in Biotechnology (Marcel Dekker, Inc), Juan A Asenjo, ed, 1990.

The invention is further explained in the following examples. The examples do not limit the invention in any way. They merely serve to clarify the invention.

### EXAMPLES

### Example 1

### Factor V Purification Based on Research-Grade Protocol Described by Bos et al. (Comparative Example)

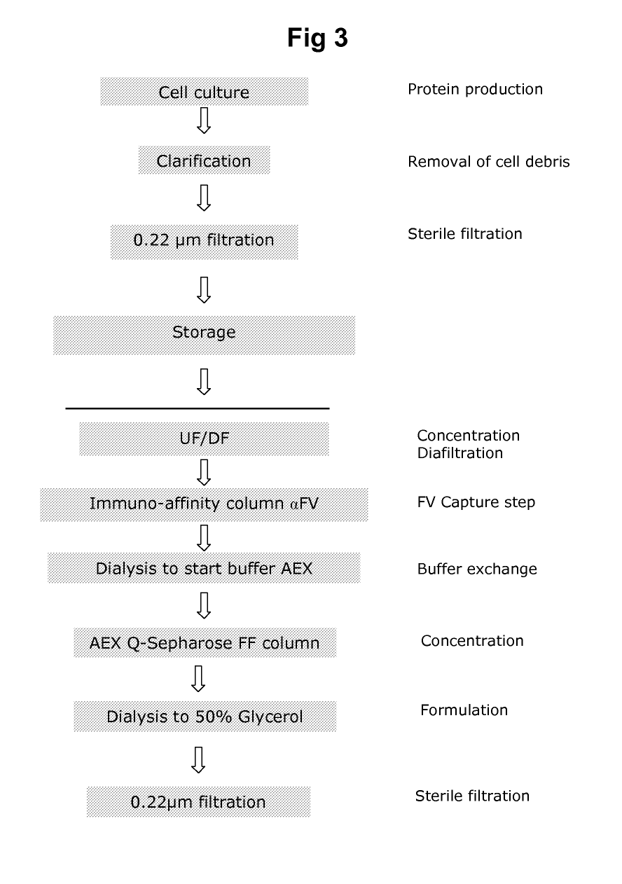

Wild-type Factor V-L/C was recombinantly produced in adherently cultured PER.C6®-FV-L/C wt cells, in roller bottles (production medium: DMEM+2.5% heat-inactivated calf serum). The protocol described by Bos et al. was implemented in a slightly adapted form. A process flow diagram is represented on FIG. 3. On day 1, an Ultrafiltration/Diafiltration (UF/DF) step with a 100 kD 0.1 sq.m Pellicon-2 membrane from Millipore was implemented as alternative to the concentration step by Hemoflow F5 (Fresenius, Bad Homberg, Germany) described by Bos et al. During the UF/DF process, the harvest was concentrated ten times over a 0.3 m² 100 KD Millipore Pellicon 2 membrane (Cat#:P2C100C01) and the concentrated material was dialyzed against 3 volumes of buffer. On day 2, the immuno-affinity was performed using standard chromatography equipment columns/Fast Performance Liquid Chromatography) instead of using the αFV-NHS (N-hydroxysuccinimide) sepharose affinity resin from GE-Healthcare and was followed by dialysis to binding buffer (for anion exchange), which was performed overnight. On day 3, anion exchange chromatography was performed using Q-sepharose FF resin from GE Healthcare, again followed by dialysis performed overnight to the 50% glycerol-based storage buffer. On day 4, the material was sterile filtered, aliquots were made and the material was stored at −20° C.

Factor V was obtained with this four-day purification process. The presence of Factor V after purification was confirmed by SDS-page analysis (not shown). The overall recovery of the process was approximately 10-20% (based on ELISA).

This protocol, which is suitable for research batch productions, has two main drawbacks: the long process time and the presence of non-scalable process steps, such as dialysis. Moreover, it includes a harsh elution step of the immuno-affinity column at pH10, which has a deleterious effect on product quality and may also reduce the life span of the anti-FV antibodies (bound to the column), leading to an overall increase of the cost of goods. Harsh elution steps are preferably avoided in large-scale operations.

### Example 2

### Factor V Purification Process According to the Invention (Anion Exchange Followed by Immuno-Affinity)

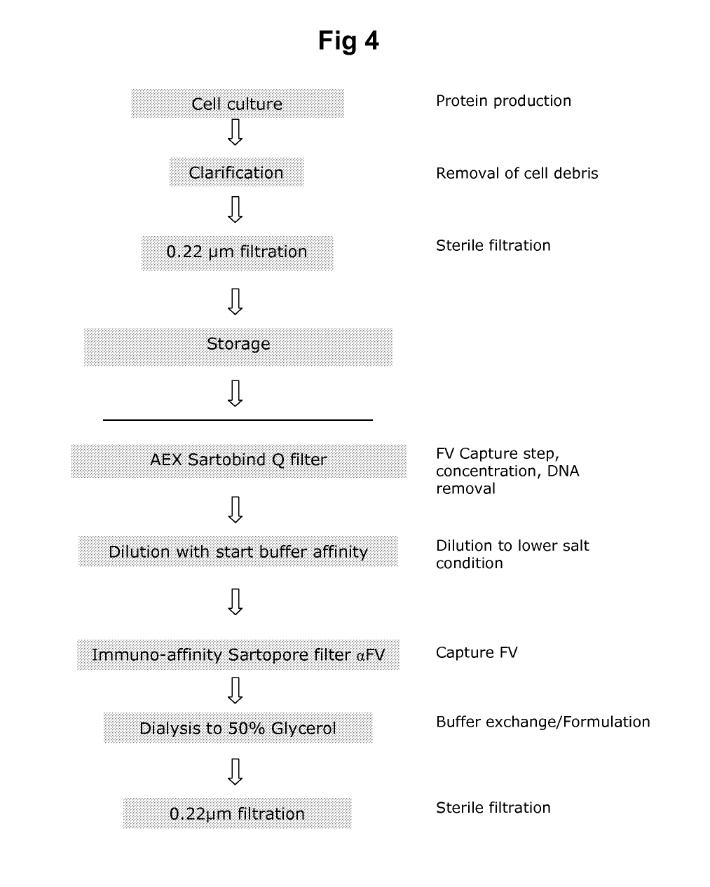

The anion exchange step and immuno-affinity step were reversed, as compared to the process described by Bos et al. Herewith, the intermediate dialysis (part of the UF/DF step) could be omitted. In addition, the resin-containing columns (of the anion exchange step and the immuno-affinity step) were replaced by filters, which allowed using higher flow rates, resulting in a reduced process time. A process flow diagram is represented on FIG. 4.

The adapted process started on day 1 with an anion exchange step using Sartobind Q filters (10K-15-25 Q filter with a bed volume of 280 ml from Sartorius) in binding mode. Elution of the Factor V was performed with 0.6 M NaCl buffer containing 10% glycerol, which gave the additional advantage of having DNA attached to the filter during elution and, thus, a cleaner Factor V preparartion for further purification. In order to remove DNA from the column, a buffer with increased ionic strength (higher NaCl concentration) was needed.

The eluate fraction of the anion exchanger was diluted four times with the start buffer of the immuno-affinity step in order to reduce the salt concentration, which aids in maximal binding of Factor V to the immuno-affinity matrix. Subsequently, the process continued on day 1 or day 2 with an immuno-affinity step using specific anti-FV antibody coupled to Sartobind Epoxy membranes (Sartobran 150 epoxy filter with a bed volume of 140 ml from Sartorius) followed by elution at pH10 with a buffer containing 50% ethylene glycol. Finally, a buffer exchange to the 50% glycerol-based storage buffer was performed.

Factor V was obtained with this two-day purification process. The presence of Factor V after purification was confirmed by SDS-page analysis (not shown). The overall recovery of the process was approximately 10-20% (based on ELISA), which was similar to the process disclosed in Bos et al.

Surprisingly, reversing the order of the anion exchange step and the immuno-affinity step resulted in similar process yields and a reduced process time (from 4 days to 2 days). An additional process improvement was the removal of host cell DNA (during the anion exchange step) prior to the immuno-affinity chromatography step, which led to less impurities/DNA loaded on the immuno-affinity filter. As a result, the filter is less likely to clog, and the number of cycles that the immuno-affinity membranes can be used for is increased, which, in turn, positively influences the cost of goods of the Factor V purification process.

### Example 3

### Factor V Purification Process According to the Invention (Use of Chromatographic Monoliths)

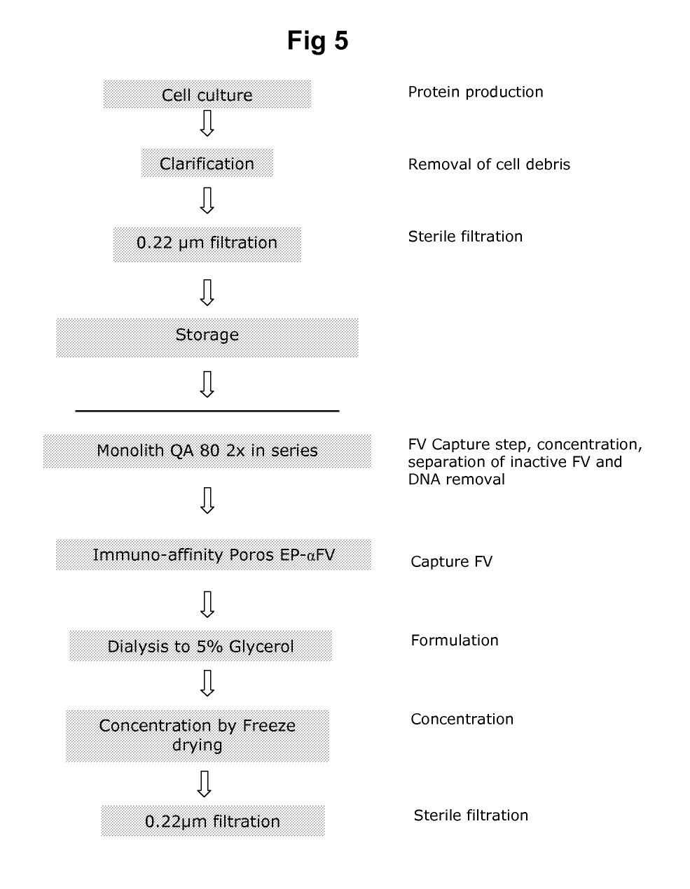

The purification protocol disclosed in Example 2 was modified herein by using a chromatographic monolith instead of a filter during the anion exchange step and a column containing a cross-linked polystyrene-divinylbenzene matrix to which epoxide functional groups are bound (POROS® matrix) instead of a filter during the immuno-affinity step. A process flow diagram is represented on FIG. 5.

The anion exchange step was performed using chromatographic monoliths containing quaternary amine groups (Monolith QA 80 ml from BIA separations with two columns mounted in series). The start buffer (binding buffer) consisted of 20 mM Tris.HCl, 200 mM NaCl, 1 mM CaCl₂ and 10% Glycerol (pH 7.4). It was observed that when applying a two-step gradient, two eluate fractions were obtained. Surprisingly, the first eluate fraction, which was obtained with elution buffer 1 (360 mM NaCl) contained active Factor V. The second eluate fraction, which was obtained with elution buffer 2 (560 mM NaCl), contained inactive Factor V. Apparently, the high resolution of the monolith allowed for the exclusion of a non-active (possibly degraded or incompletely processed) fraction of Factor V. Herewith, a method was provided to obtain a Factor V preparation with an increased activity. DNA was also removed during this step.

The activity, which can be translated into the ability to induce clot formation, was tested in a clot activity assay using FV-deficient human plasma. The eluate fractions were added to FV-deficient plasma (Dade Behring, Germany), employing normal human plasma as reference. Clotting was induced with Innovin® (Dade Behring) or with Thromborel S (Dade Behring). Pooled plasma was again used as a standard. One unit of Factor V activity was similar to the amount of FV in 1 mL of normal plasma (±8 μg/mL).

The clot activity assay, which is based on the correlation between the Factor V concentration and the rate of clot formation, allows for determining the Factor V concentration in the eluate fractions. The specific clot activity or RATIO (the ratio of active Factor V to the total Factor V fraction) was obtained by dividing the Factor V concentration (determined by the clot activity assay) with the total Factor V concentration (determined by ELISA). Table 1 shows the results of the clot activity assay for both the eluate fractions of Factor V obtained from the anion exchange step (using a monolith).

TABLE 1
ELISA and clot activity assay

| | ELISA | CLOTTING | |
|---|---|---|---|
| | (μg/ml) | ASSAY (μg/ml) | RATIO |
| Eluate fraction 1 | 57* | 41* | 0.7 |
| Eluate fraction 2 | 16* | <Detection level | NA |

*Average of three samples

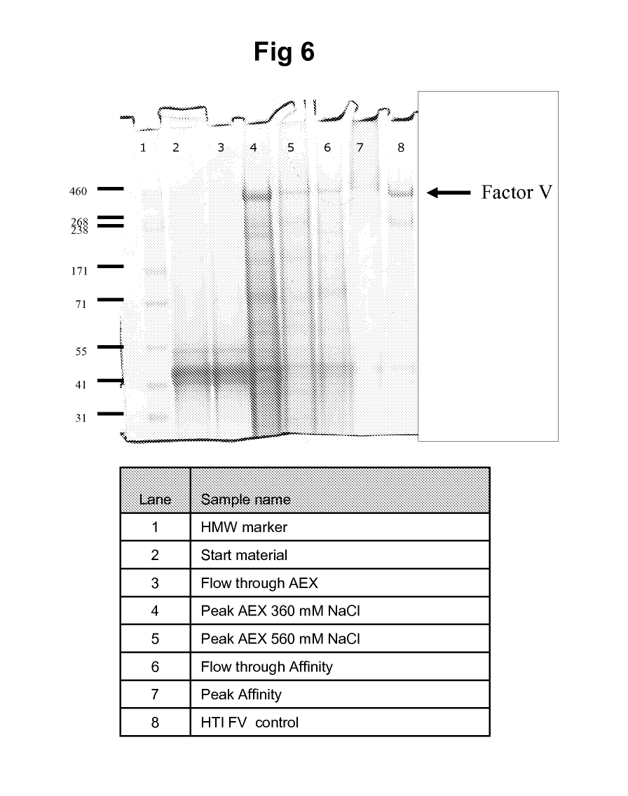

Eluate fraction 1 contained a 70% active Factor V preparation while Eluate fraction 2 had an undetectable activity. Factor V, which was clearly present in both eluate fractions (SDS-Page on FIG. 6), was surprisingly separated in an active and an inactive form with the use of a monolith.

An additional advantage of the monolith was the lower dead volume compared with the filter membranes resulting in a reduced dilution of the sample.

Importantly, the monolith can be run at equally high flow rates compared to the membranes. Due to the lower salt concentration of the first eluate fraction of the monolith, this sample does not have to be diluted before loading it onto the affinity column, thus reducing the loading time of the affinity column and keeping the sterility safe.

The immuno-affinity step was performed using an anti-FV antibody containing matrix based on 50 μm flowthrough particles made of a cross-linked polystyrene-divinylbenzene to which epoxide functional surface groups are bound, from Applied Biosystems (POROS® matrix).

Unexpectedly, the POROS® matrix has the advantage of less aspecifically binding impurities (compared to filter), resulting in a higher purity of the final preparation. Also, the dead volumes in the POROS® matrix are smaller then those in the filters and allow for higher peak concentrations. The specific binding and low dead volumes resulted in higher recovery yields. Moreover, the presence of rigid beads with big pore sizes in the POROS® matrix allowed for high flow capacity (similar to the flow in filters).

The Factor V was eluted from the column using high-salt treatment (buffer containing 2 M NaCl, 50% Ethyleneglycol with a pH of 7.4), replacing the previously used pH10 elution buffer.

Optionally, if desired, the preparation can either be further concentrated using conventional methods prior to buffer exchange to the final 50%-glycerol-based storage buffer or buffer exchanged to a 5%-glycerol-based buffer and then freeze dried to obtain a final concentrated sample with a concentration of 50% glycerol.

The currently described protocol appeared to be very well suited for scale-up. Moreover, the current protocol is capable of delivering an active preparation of recombinant Factor V-L/C after two process steps with an over-all recovery of about 30% (based on ELISA), which is higher then the process disclosed in Bos et al.

### REFERENCES

Bertina R. M., B. P. Koeleman, T. Koster, F. R. Rosendaal, R. J. Dirven, H. de Ronde, P. A. van der Velden, and P. H. Reitsma. Mutation in blood coagulation factor V associated with resistance to activated protein C. Nature 369 (6475):14-5 (1994).
Bos M. H., D. W. Meijerman, C. van der Zwaan, and K. J. Mertens. Does activated protein C-resistant factor V contribute to thrombin generation in hemophilic plasma? Thromb. Haemost. 3:522-30 (2005).
Chan W. P., C. K. Lee, Y. L. Kwong, C. K. Lam, and R. Liang. A Novel Mutation of Arg306 of Factor V Gene in Hong Kong Chinese. Blood 91:1135-39 (1998).
Nesheim M. E., K. H. Myrmel, L. Hibbard and K. G. Mann. Isolation and Characterization of single chain bovine factor V. JBC Vol. 254, Issue 2, 508-517, January 1979.
van der Neut Kolfscholten M., R. J. Dirven, G. Tans, J. Rosing, H. L. Vos, and R. M. Bertina. The activated protein C (APC)-resistant phenotype of APC cleavage site mutant of recombinant factor V in a reconstituted plasma model. Blood Coagul. Fibrinolysis 13:207-215 (2002).
Svensson P. J., and B. Dahlback. Resistance to activated protein C as a basis for venous thrombosis. NEJM 330:517-522 (1994).
Williamson D., K. Brown, R. Luddington, C. Baglin, and T. Baglin. Factor V Cambridge: a new mutation (Arg306-->Thr) associated with resistance to activated protein C. Blood 91 (4):1140-44 (1998).

1. A method for purifying Factor V from a biological fluid, said method comprising in the given order the steps of:
a) Binding factor V to an anion exchanger;
b) Washing the anion exchanger with a first solution to remove contaminants;
c) Eluting Factor V;
d) Specifically binding factor V to a matrix containing anti-Factor V antibodies;
e) Washing said matrix containing anti-Factor V antibodies with a second solution to remove contaminants; and
f) Eluting Factor V.

2. The method according to claim 1, wherein the anion exchanger in step a) is a filter.

3. The method according to claim 1, wherein the anion exchanger in step a) is a chromatographic monolith containing quaternary amine groups.

4. The method according to claim 1, wherein the elution in step c) is performed by treating the anion exchanger with a buffer containing between 0.3 and 1 M NaCl buffer.

5. The method according to claim 1 wherein the matrix of step d) is an immuno-affinity capture filter membrane.

6. The method according to claim 1, wherein the matrix of step d) is a cross-linked polystyrene-divinylbenzene matrix to which Epoxide functional groups are bound.

7. A method for purifying Factor V from a biological fluid, the method comprising:
utilizing a chromatographic monolith containing quaternary amine groups.

8. A method for separating active Factor V from inactive Factor V, the method comprising:
utilizing a chromatographic monolith containing quaternary amine groups.

9. The method according to claim 8, wherein the active form of Factor V is at least two times as active as the inactive form of Factor V in a clot activity assay.

10. The method according to claim 1, wherein Factor V has been recombinantly expressed.

11. The method according to claim 1, wherein Factor V is an APC-resistant Factor V mutant.

12. A scalable method for removing Factor V from a liquid containing Factor V, the method comprising:
purifying the liquid with a chromatographic monolith anion exchanger containing quaternary amine groups to concentrate Factor V thereon;
removing contaminants therefrom with a solution;
eluting Factor V from the chromatographic monolith anion exchanger by treating the chromatographic monolith anion exchanger with a buffer;
specifically binding the thus eluted Factor V to a matrix having anti-Factor V antibodies;
washing the matrix containing bound anti-Factor V antibodies to remove contaminants therefrom; and
eluting Factor V from the matrix.

13. The method according to claim 12, wherein the matrix is an immuno-affinity capture filter membrane.

14. The method according to claim 12, wherein the matrix is a cross-linked polystyrene-divinylbenzene matrix to which epoxide functional groups are bound.

15. The method according to claim 14, wherein the active form of Factor V is at least two times as active as the inactive form of Factor V in a clot activity assay.

16. The method according to claim 12, wherein Factor V is an APC-resistant Factor V mutant.
